(19) United States
(12) Patent Application Publication
(10) Pub. No.: US 2017/0172453 A1
(43) Pub. Date: Jun. 22, 2017

(54) SYSTEM AND METHOD FOR QUANTITATIVE IMAGING USING MULTIPLE MAGNETIZATION PATHWAYS

(71) Applicant: BRIGHAM AND WOMEN'S HOSPITAL, INC., Boston, MA (US)

(72) Inventors: BRUNO MADORE, Stockdorf, MA (US); Cheng-chieh CHENG, Taipei (TW)

(21) Appl. No.: 15/128,302

(22) PCT Filed: Mar. 31, 2015
(86) PCT No.: PCT/US2015/023598

§ 371 Date: Sep. 22, 2016

Related U.S. Application Data
(60) Provisional application No. 61/972,653, filed on Mar. 31, 2014.

(51) Int. Cl. A61B 5/055; G01R 33/50; G01R 33/24; A61B 5/00

(52) U.S. Cl. CPC A61B 5/0555; A61B 5/742; G01R 33/50; G01R 33/243

(57) ABSTRACT

A system and method for quantitative imaging using a magnetic resonance imaging (MRI) system is disclosed. The method includes generating a steady-state pulse sequence on the MRI system, performing the pulse sequence to acquire magnetic resonance (MR) signals from a volume of the subject with two or more scans, representing physical parameters of the subject in relation with the MR signals through equations, generating values of the physical parameters of the subject, and generating a report indicating the physical parameters. The pulse sequence can sample two or more magnetization pathways of MR signals and at least one scan parameter of the pulse sequence changes from scan to scan.

### CROSS REFERENCE

This application is based on, claims priority to, and incorporates herein by reference in its entirety, U.S. Provisional Patent Application Ser. No. 61/972,653, filed on Mar. 31, 2014, and entitled “Quantitative MRI and Synthetic Image Generation.”

### STATEMENT REGARDING FEDERALLY SPONSORED RESEARCH

This invention was made with government support under R01CA149342 awarded by the National Institutes of Health. The government has certain rights in the invention.

### BACKGROUND

Magnetic resonance (MR) exams can last many minutes. During this time, the anatomy of interest may be repeatedly imaged to generate a variety of tissue contrasts. The proper visualization and characterization of tumors and other pathologies often require several different tissue contrasts. Thus, many clinical exams can last 45 minutes or longer.

Besides imaging of various tissue contrasts, another group of clinical MR tools is referred to as quantitative imaging, which maps the quantitative MR parameters, such as T₁ or T₂, of a subject. There are well-established MR methods that can map one MR parameter at a time. To evaluate multiple parameters using those methods, the exam times extend to at least the duration required to perform independent studies of all desired parameters and, as such, at least the duration of many current clinical exams.

It would be desirable to have a system and method for reducing scan times when generating parametric maps of multiple parameters and images of the subject of various interested contrasts.

### SUMMARY

The present disclosure overcomes the aforementioned drawbacks by providing a system and method that uses two or more scans of a steady-state pulse sequence to sample two or more magnetization pathways, along which the transverse magnetization immediately after and before an RF field evolves when a train of RF pulses are applied. Physical parameters are represented in linear or single-variable equations in relation with the magnetic resonance (MR) signals. By solving those equations, the physical parameters can be derived. These parameters can be used to generate parametric maps or calculate images of desired contrasts with greater efficiency than traditional methods.

In accordance with one aspect of the disclosure, a method is provided for quantitative imaging of a subject using a magnetic resonance imaging (MRI) system. The method includes generating, using the MRI system, a steady-state pulse sequence to sample two or more magnetization pathways of MR signals. The method also includes performing the pulse sequence to acquire the MR signals from a volume of the subject with two or more scans. A scan refers to repetitions of the pulse sequence enough times so to scan the volume of the subject once. The method also includes assembling a series of linear equations or single-variable equations using the MR signals to represent physical parameters of the subject in relation with the MR signals, generating values of the physical parameters of the subject by solving the linear equations or single-variable equations, and generating a report indicating the physical parameters.

In accordance with another aspect of the disclosure, an MRI system is provided. The MRI system includes a magnet system configured to generate a polarizing magnetic field about at least a portion of a subject arranged in the MRI system and a magnetic gradient system including a plurality of magnetic gradient coils configured to apply at least one magnetic gradient field to the polarizing magnetic field. The MRI system also includes a radio frequency (RF) system configured to apply an RF field to the subject and to receive MR signals therefrom and a computer system. The computer system is programmed to control the gradient system and the RF system to generate a steady-state pulse sequence to sample two or more magnetization pathways of the MR signals. The computer system is also programmed to control the gradient system and the RF system to perform the pulse sequence to acquire MR signals from a volume of the subject with two or more scans. At least one scan parameter of the pulse sequence changes from scan to scan. The computer system is further programmed to assemble a series of linear equations or single-variable equations using the MR signals to represent physical parameters of the subject in relation with the MR signals and generate a report indicating the physical parameters.

In yet another aspect of the invention, methods for performing quantitative imaging of a subject vising a MRI system with one scan is provided. The method can include generating a steady-state pulse sequence on the MRI system, performing the pulse sequence to acquire MR signals from a volume of the subject with one scan, representing physical parameters of the subject in relation with the MR signals through equations, generating values of the physical parameters of the subject, and generating a report indicating the physical parameters. The pulse sequence can sample two or more magnetization pathways of MR signals. The equations representing the relation between the physical parameters and the MR signals are linear equations or single-variable equations. The physical parameters include T₂, T₂*, and frequency offset.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an example of a magnetic resonance imaging (MRI) system configured to employ the present disclosure.

FIG. 2 is a flow chart setting forth the steps of one, non-limiting example of a method for generating a report of parameter values according to the present disclosure.

FIG. 3 is a diagram of transverse magnetization when a train of radio frequency pulses have been applied in accordance with the present disclosure.

FIG. 4 is a graphic illustration of an exemplary pulse sequence used to sample multiple magnetization pathways in accordance with the present disclosure.

### DETAILED DESCRIPTION

A system and method is disclosed herein that allows magnetic resonance (MR) images of any desired contrast to be calculated, rather than scanned, based on MR data acquired in only a few minutes. The information obtained through a pair of relatively-short scans can be used to generate three-dimensional (3D) maps of, for example, all of the main MR physical parameters that are used to determine contrast: relaxation times T₁, T₂ and T₂*; equilibrium magnetization M₀; flip angles; and frequency offset Δf. Note a scan refers to repetitions of a pulse sequence enough times to scan a volume of a subject once. While a single scan is sufficient to evaluate parameters-such as T₂, T₂* and the offset frequency, a second scan may be needed to find other parameters, such as T₁, flip angle and M₀. The flip angle map is expected to be spatially smooth and, for this reason, the second and later scans can be acquired with reduced spatial resolution, to further reduce scan time. These parameter maps can be directly displayed, leading to quantitative MR imaging where each pixel in the images has physically meaningful units. With the fast acquisition of clinically-adequate amount of information, the system and method allows the cost of MRI exams to be low enough to be used for screening and helps MR imaging transition from being mostly qualitative to quantitative.

MR imaging to this day mostly generates non-quantitative grayscale images. While contrast can be manipulated to produce clinically-useful images, one rarely obtains maps of the actual physical parameters that give rise to contrast. As imaging strategies and acceleration techniques continue to evolve, MRI images may become quantitative in nature, with values that have actual units and physical meaning. Some examples of physical parameters used to determine contrast include: equilibrium magnetization M₀—source of all MR signals, flip angles—the angle that magnetization is rotated from longitudinal axis to the transverse plane and vice-versa, the spin-spin time constant T₂—irreversible decay in the transverse plane, T₂*—combination of reversible and irreversible decay, spin-lattice time constant T₁—signal growth along the longitudinal axis, and frequency offset Δf. M₀ may include the sensitivity of the receive coil array.

The system and method disclosed herein allows a fast, robust quantitative imaging where MR parameters are calculated from the acquired signal in a way that is fairly independent from each other and the evaluation of them involves linear or single-variable equations. It is thus more reliable and robust, compared to other attempts, where the evaluation may involve numerically solving larger and non-linear equations of many or all parameters at once.

The system and method disclosed herein can be used to improve the accuracy of the estimated parameters further by treating the flip angle as unknown and to be evaluated rather than a known user-input quantity. This is because the flip angle actually experienced at a given location in the subject is generally not equal to the one prescribed by the user. The flip angle progressively decreases from a value close to the user-prescribed one in the center of a 3D excited volume to near zero at the edges of the volume. This spatial variability is also subject-dependent. If not accounted for, such variability in flip angle would cause errors in the parametric estimation.

With the values of those physical parameters, MR images of any desired contrast can be synthesized through signal equations well-known to a person skilled in the art. Clinical MR exams have a relatively long duration in part because the same anatomy is imaged multiples times to achieve different contrasts. However, the MRI exam can be much shorter with the system and method disclosed herein because images of all desired contrasts can be quickly calculated as the main parameters determining the contrasts have been estimated from the MR signals acquired with much shorter scan time. New types of contrasts and grayscales, including some physically impossible to generate directly with MRI, can even be generated.

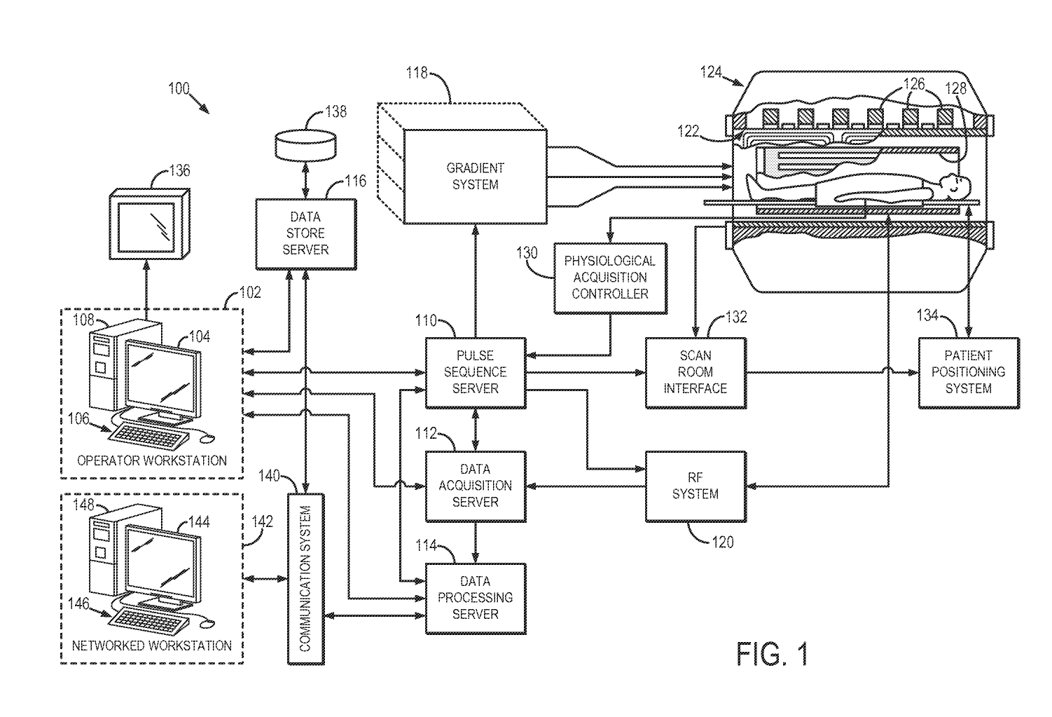

Referring particularly to FIG. 1, an example of an MRI system 100 is illustrated. The MRI system 100 includes a workstation 102 having a display 104 and a keyboard 106. The workstation 102 includes a processor 108 that is commercially available to run a commercially-available operating system. The workstation 102 provides the operator interface that enables scan prescriptions to be entered into the MRI system 100. The workstation 102 is coupled to four servers: a pulse sequence server 110; a data acquisition server 112; a data processing server 114; and a data store server 116. The workstation 102 and each server 110, 112, 114, and 116 are connected to communicate with each other.

The pulse sequence server 110 functions in response to instructions downloaded from the workstation 102 to operate a gradient system 118 and a radiofrequency (RF) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128 (or specialized RF coils such as a head and neck or a knee coil, for example).

RF excitation waveforms are applied to the RF coil 128, or a separate local coil, such as a head coil, by the RF system 120 to perform the prescribed magnetic resonance pulse sequence. Responsive MR signals detected by the RF coil 128, or a separate local coil, are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays.

The RF system 120 also includes one or more RF receiver channels.

The whole body RF coil 128 or one or more local coils or coil arrays can be used to receive RF signals. Each RF receiver channel includes an RF preamplifier that amplifies the MR signal received by the coil to which it is connected, and a detector that detects and digitizes the received MR signal. The magnitude of the received MR signal may be represented at any sampled point by the square root of the sum of the squares of the imaginary I and real Q components:

M=\sqrt{I²+Q²}

and the phase of the received MR signal may also be determined:

$$ϕ = {tan}^{- 1} ( \frac{Q}{I} ) .$$

The pulse sequence server 110 also optionally receives patient data from a physiological acquisition controller 130. The controller 130 receives signals from a number of different sensors connected to the patient, such as electrocardiograph (ECG) signals from electrodes, or respiratory signals from a bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processing server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled. In all these examples, the data acquisition server 112 acquires MR data and processes it in real-time to produce information that is used to control the scan.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory cache (not shown), from which they may be output to operator display 104 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network or communication system 140 to other facilities that may include other networked workstations 142.

The communication system 140 and networked workstation 142 may represent any of the variety of local and remote computer systems that may be included within a given clinical or research facility including the system 100 or other, remote location that can communicate with the system 100. In this regard, the networked workstation 142 may be functionally and capably similar or equivalent to the operator workstation 102, despite being located remotely and communicating over the communication system 140. As such, the networked workstation 142 may have a display 144 and a keyboard 146. The networked workstation 142 includes a processor 148 that is commercially available to run a commercially-available operating system. The networked workstation 142 may be able to provide the operator interface that enables scan prescriptions to be entered into the MRI system 100.

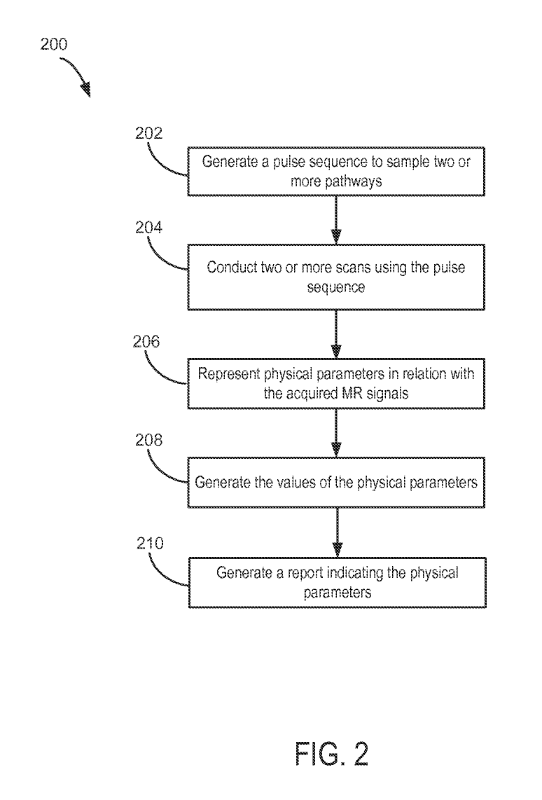

The system and method disclosed herein can be used with an MRI system, such as the above-described system 100, to sample multiple magnetization pathways for quantitative imaging. Referring to FIG. 2, a flowchart 200 of an example method implemented according to the present disclosure is provided. In step 202, a pulse sequence is generated to sample two or more pathways. In step 204, two or more scans are conducted using the pulse sequence generated in step 202, and MR signals are acquired. In step 206, physical parameters in relation with the acquired MR signals in step 204 are represented through linear or single-variable equations. The physical parameters can be T₁, T₂, T₂*, frequency offset Δf, flip angles, and equilibrium magnetization M₀. In step 208, the values of the physical parameters are generated by solving those equations. In step 210, a report indicating the physical parameters can be generated. Because these physical parameters can be generated using the equations, rather than repetitive samplings, the above-described process can be more efficient and effective than traditional methods and can give rise to reports or images highlighting physical parameters not readily available using traditional methods of quantitative MR imaging.

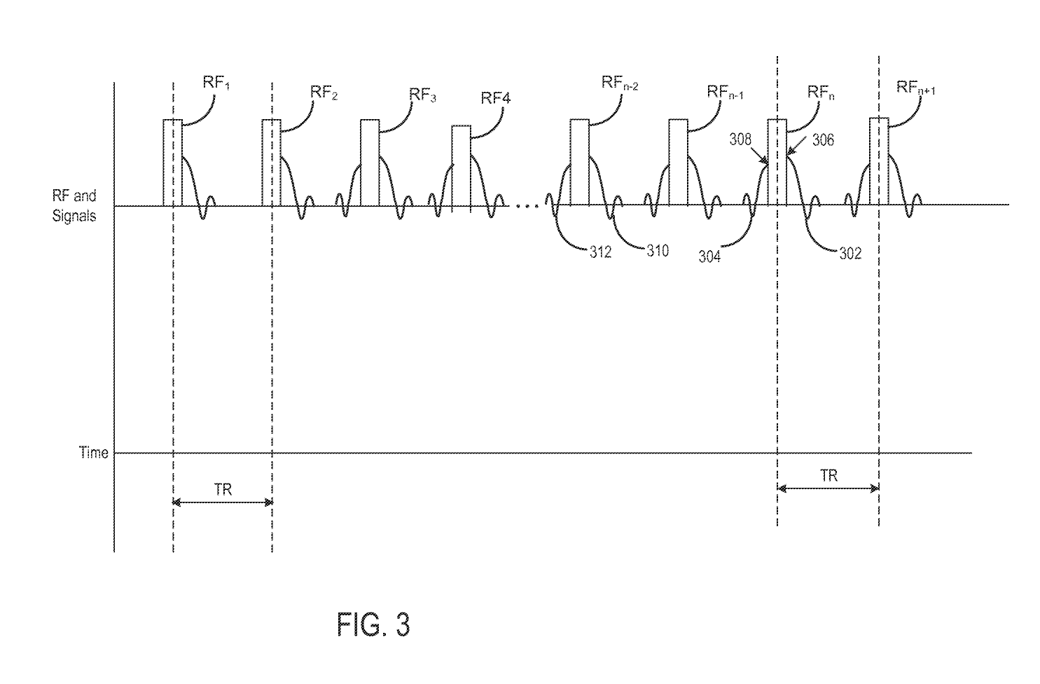

Referring to FIG. 3, a pictorial depiction of the transverse magnetization is provided for the case where a train of RF pulses are applied, typically with repetition time (TR)<T₂. After many RF pulses have been applied a steady-state situation is reached whereby the same magnetization is generated periodically, for example magnetization 302 may be essentially equal to 310 and magnetization 304 may be essentially equal to 312. The magnetization 306 immediately after RFₙ is referred to as F⁺ and the magnetization 308 immediately before RFₙ is referred to as F⁻. Both F⁺ and F⁻ consist of many different components that can be independently sampled, referred to as Fₖ⁺ and Fₖ⁻, respectively, where k is the pathway number. Different pathways have different weightings in terms of T₁, T₂ and T₂* and are found at different locations in k-space. A steady-state unbalanced sequence can be used: It is a steady-state sequence because RF spoiling is not employed and magnetization is allowed to endure from one TR interval to the next, and it is unbalanced because all gradients waveforms do not add up to a zero area. This unbalanced gradient can be used to determine how far apart different magnetization pathways are in k-space, and where to find them.

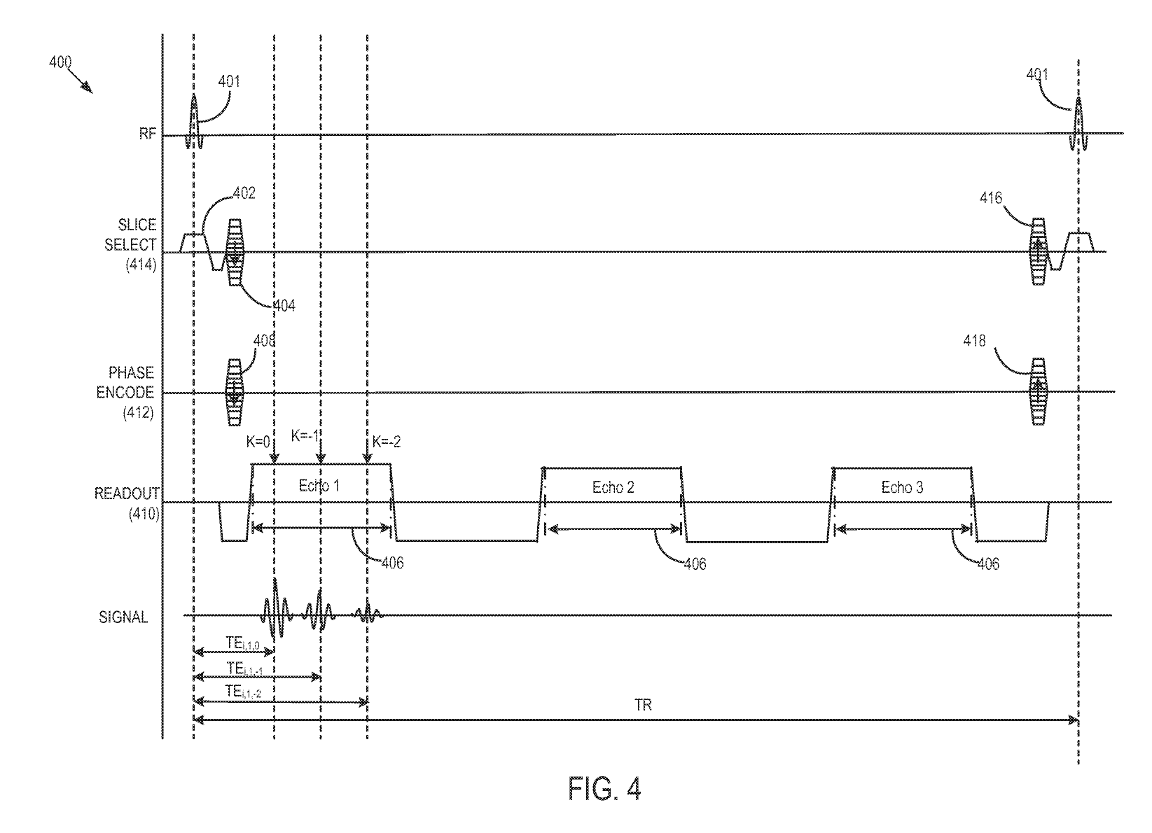

The system and method disclosed herein uses a pulse sequence that can sample individual pathways. Referring to FIG. 4, a diagram of an example pulse sequence is provided. This is an unbalanced steady-state fly-back sequence. Signals from different pathways tend to line in the order of the pathway numbers, e.g., +2, +1, 0, −1, −2. In FIG. 4, both the slice-select 414 and phase-encode 412 waveforms add up to a zero total area, and the readout 410 waveform is unbalanced. For this reason different pathways are lined at different location along the readout direction and can be harvested in turn, as part of an extended readout window 406. So, individual pathways can be sampled by changing the timing and duration of a readout window 406. Individual pathways can be thought of as replicas of the k-space signal which is typically found near k-space center. These replica differ from each other in many ways, mostly due to differing T₁, T₂ and T₂* weightings, and the k-space distance between neighboring replicas is given by the gradient unbalance, e.g., the unbalanced readout waveform 410 in FIG. 4. Typically, the strength of these replicas tends to quickly decrease with increasing pathway number, |k|, and with distance from k-space center. Such replicas always exist with unbalanced steady-state sequences, but these are typically designed to only sample the 0^{th} pathway, the one normally located nearest from k-space center. By changing the sampling scheme, in the case of FIG. 4 by traveling further than usual in k-space using a longer than usual readout window, extra pathways beyond the 0^{th} can be reached and sampled. The 0^{th} pathway is typically found near k-space center while the +k^{th} and −k^{th} pathways are found away from k-space center in opposite directions. The depicted pulse sequence samples pathways 0, −1, and −2; however, this is a non-limiting example and other pathways can be sampled.

Still referring to FIG. 4, the pulse sequence 400 includes an RF train 401, a slice-select gradient 414, a phase encoding gradient 412, and readout gradients 410. The time between the adjacent RFs is TR. The slice select and phase-encoding gradients can be in any pattern used for a certain k-space trajectory. Depicted is a 3D gradient echo acquisition with a slab-select gradient 402 to select a slab, slab-encoding gradient 404, and a phase encoding gradient 408 to sample k-space positions in the kz and ky directions, respectively. Slab-selective RF pulses are not required, but can be used to limit the extent of the imaged field-of-view and thus reduce scan time. Two gradients 416 and 418 act as the rephasers of the slab-encoding gradient 404 and phase encoding gradient 408, respectively (note the opposite directions of the arrows in 404 and 416, 408 and 418). The kx direction is sampled by the readout gradients 410. This pulse sequence is designed to acquire a train of three echoes (echoes 1, 2, and 3). The subject can be imaged with two or more scans, where the pulse sequence 400 is repeated with different scan parameters, such as TR or the RF flip angle. The scans can be sequential, where a scan is conducted with a different set of scan parameters after one scan is completed. The scans can also be interleaved, where the pulse sequence is run with one set of scan parameters during one TR and a different set of parameters during the next TR. The scans may have different spatial resolution. No matter whether the scans are sequential or interleaved, the pulse sequence 400 can be performed such that there is no time lag between different scans.

The system and method disclosed herein can use a steady-state pulse sequence, which means that RF spoiling is not used to destroy the transverse magnetization from previous TR periods. Such pulse sequence may create a vast number of different magnetization pathways which coexist and may be individually sampled. The pulse sequence 400 depicted in FIG. 4 is designed to sample three pathways: the 0^{th}, −1^{st} and −2^{nd} pathways. Each pathway is sampled at a few different moments during the TR period, i.e., at a few different echo times, as shown in FIG. 4. Specifically, TE_{i,j,k} represents the echo time associated with the j^{th} echo for the k^{th} pathway during the i^{th} scan. In the time interval [0, TR], where t=0 is defined as the moment an RF pulse is applied, the signal S_{i,k}(t) varies as:

$${S}_{i , k} ( t ) ∝ \{ \begin{matrix}{e}^{- ( {R}_{2} + {R}_{2}^{′} ) t} , & k \geq 0 \\ {e}^{- ( {R}_{2} - {R}_{2}^{′} ) t} , & k < 0\end{matrix} ; \qquad [ 1 ]$$

where R₂ and R₂′ represent irreversible and reversible decay, respectively, such that T₂=1/R₂ and T₂*=1/(R₂+R₂′). Because signal for negative pathways is similar to a spin-echo on its way to formation, reversible decay is in the process of being corrected and R₂′ appears with a minus sign in Eq. 1 for |k|<0. On the other hand, for positive pathways, both R₂ and R₂′ are combined to give rise to T₂* decay, as seen in Eq. 1 for k≧0. The signal is sampled only when an echo is formed, which means that only the values S_{i·k}(TE_{i·k·k}) are known. Equation 1 can be converted into linear equations:

$$ln ( \vert {S}_{i , k} ( {TE}_{i , j , k} ) \vert ) = \{ \begin{matrix}ln ( \vert {S}_{i , k} ( 0 ) \vert ) - {TE}_{i , j , k} ( {R}_{2} + {R}_{2}^{′} ) , & k \geq 0 \\ ln ( \vert {S}_{i , k} ( 0 ) \vert ) - {TE}_{i , j , k} ( {R}_{2} - {R}_{2}^{′} ) , & k < 0\end{matrix} . \qquad [ 2 ]$$

These linear equations can used to evaluate R₂ and R₂′ with data acquired with multi-echo sequences such as the one shown in FIG. 4. With N_{TE,i} the number of echoes sampled in the i^{th} scan, N_{P} the number of sampled pathways and N_{S} the number of scans, Eq. 2 gives rise to

$${\sum }_{i} ( {N}_{{TE}_{j}^{i}} {N}_{p} )$$

linear equations with N_{S}×N_{P}+2 unknowns. For R₂ and R₂′ to be separable, at least one of each pathway type, k≧0 and k<0, is sampled. Evaluating the flip angle, T₁, and M₀ may require at least two scans (N_{S}=2) and three sampled pathways (N_{P}=3). All of these acquired data can be included to solving R₂ and R₂′ through Eq. 2 and, then T₂ and T₂* if needed.

In a case where R₂, R₂′ and the frequency offset Δf are the parameters of primary interest, a single scan is sufficient to evaluate them: With a single scan, N_{S}=1, and two pathways, N_{P}=2, and two echo times, N_{TE}=2, one would get 4 equations, which may be sufficient to solve for 4 unknowns. A larger number of echoes, N_{TE}>2, would allow the number of equations to be increased and the overall conditioning of the problem improved while keeping the number of unknowns unchanged: S₁(0), S₂(0), R₂ and R₂′. The frequency offset can readily be found from phase variations from echo to echo, in all NP pathway signals. While this simpler implementation would not provide all of the parameters sought here, it represents a simpler and faster one-scan approach to evaluate a subset of them, such as R₂, R₂′ and Δf.

The choice of pathways to be sampled may be dictated, in part, by Eq. 2. The 0^{th} and −1^{st} pathways are desired because they typically have the strongest signal among all k≧0 and k<0 pathways, respectively. As for a third pathway, the −2^{nd} is generally preferred over the +1^{st} because it brings greater stability to the solution of Eq. 2. Indeed, the (R₂+R₂′) dependency may be more readily captured in strong k=0 signals than the (R₂−R₂′) dependency in the weaker k=−1 signals and, for this reason, the sampling of an extra negative pathway can prove beneficial to the conditioning of Eq. 2. However, the method as disclosed herein can accommodate cases where a second k≧0 pathway is sampled in place of a second k<0 pathway, as long as at least one of each kind is obtained, i.e., at least one k≧0 pathway and at least one k≦0 one are sampled.

The concept of magnetization states F and Z and relations between those states and MR parameters of interest are described in more detail before MR-related parameters beyond R₂ and R₂′ (or equivalently, T₂ and T₂*) are solved. Specifically, Fₖ and Zₖ denote the transverse and longitudinal magnetization for pathway level k. Superscripts −, + and [symbol] are used to distinguish between magnetization states just before, just after and a time TR after an RF pulse:

$${F}_{i , k}^{+} = {S}_{i , k} ( 0 ) ; {F}_{i , k}^{⇒} = {S}_{i , k} ( TR ) = \{ \begin{matrix}{S}_{i , k} ( 0 ) {e}^{TR ( {R}_{2} + {R}_{2}^{′} )} , & k \geq 0 \\ {S}_{i , k} ( 0 ) {e}^{TR ( {R}_{2} - {R}_{2}^{′} )} , & k < 0\end{matrix} . \qquad [ 3 ]$$

The transverse magnetization levels |F_{i,k}⁺| and | [symbol] | can be evaluated through Eq. 3 using the values for |S_{i,k}(0)|, and R₂ and R₂′ previously obtained from solving Eq. 2. The phase of the complex-valued [symbol] and F_{i,k}⁺ terms is not yet known at this point but it is needed only to evaluate the frequency offset Δf. The value of F_{i,k}⁻ is closely related to that of [symbol] ; their exact relationship depends on whether the two separate scans are interleaved or sequential manner, with or without RF chopping. Unlike the transverse magnetization F states, the longitudinal Z states usually cannot be readily sampled by a pulse sequence without interrupting the steady-state, and may have to be calculated rather than measured.

Equations that relate the F and Z magnetization states to MR parameters of interest such as T₁ and the flip angle are as follows. For example, the longitudinal states Z_{i,k}⁺ and [symbol] are linked through T₁:

$${Z}_{i , k}^{⇒} = \{ \begin{matrix}{Z}_{i , k}^{+} {e}^{- {TR}_{i} / {T}_{1}} , & \vert k \vert > 0 \\ {M}_{0} ( 1 - {e}^{- {TR}_{i} / {T}_{1}} ) + {Z}_{i , 0}^{+} {e}^{- TR / {T}_{1}} , & k = 0\end{matrix} . \qquad [ 4 ]$$

The zero and non-zeroth pathways behave differently with respect to T₁ as the latter represent dephased magnetization states decaying with T₁ while the k=0 pathway represents a magnetization vector recovering with T₁. All Z states have Hermitian symmetry with respect to k:

Z_{i,k}^{O}=Z̅_̅{̅i̅,̅-̅k̅}̅^̅{̅O̅}̅ [5];

where O is a wildcard placeholder for any one of the ‘+’, ‘−’, or [symbol] superscripts, and the bar above Z in Z̅ represents that Z̅ is the complex conjugate of Z. The Z_{i,k} and F_{i,k} states just before and after an RF pulse are related through:

$${F}_{i , k}^{+} = \{ \begin{matrix}{F}_{i , k}^{-} {cos}^{2} ( {α}_{i} / 2 - \overline{{F}_{i , - k}^{-}} {sin}^{2} ( {α}_{i} / 2 ) + {Z}_{i , k}^{-} sin ( {α}_{i} ) , & \vert k \vert > 0 \\ {F}_{i , 0}^{-} cos ( {α}_{i} ) + {Z}_{i , 0}^{-} sin ( {α}_{i} ) , & k = 0\end{matrix} . \qquad [ 6 ]$$

Like Eq. 4, Eq. 6 has a different form for the zero pathway because it corresponds to a vector, not a dephased distribution of vectors. Z_{i,k} relates to F_{i,k} states before and after an RF pulse as:

Z_{i,±k}⁺=F_{i,±k}⁻sin(αᵢ)+Z_{i,±k}⁻cos(αᵢ) [7].

Adding the +k and −k versions of Eq. 7, and using the symmetry expressed in Eq. 5, one obtains:

Z_{i,k}⁺=Z_{i,k}⁻cos(αᵢ)−(F_{i,k}⁻+F̅_̅{̅i̅,̅-̅k̅}̅⁻̅)sin(αᵢ)/2. [8].

Using expressions for both Zₖ⁻ and Z₋ₖ⁻ from Eq. 6 and the symmetry expressed in Eq. 5, a relation is obtained that involves only F states:

F_{i,k}⁺−F̅_̅{̅i̅,̅-̅k̅}̅⁺̅−F_{i,k}⁻+F̅_̅{̅i̅,̅-̅k̅}̅⁻̅=0. [9].

The cosine values cos(αᵢ) of the flip angles m are evaluated as follows. Two or more scans can be obtained with different acquisition settings to calculate the flip angles. The scans may differ in their nominal flip angles—the flip angles user prescribed (e.g., \hat{α}₁, and \hat{α}₂), or TR (e.g., TR₁, and TR₂). In fact, only the cosines of the flip angles are needed, not the flip angles themselves. The relation between cos(α_{i>1}) and α₁ can be known, for a given RF pulse design:

cᵢ=cos(αᵢ); c_{i>1})=g(α₁) [10];

where g(·) denotes the relation between c_{i>1} and α₁.

If a two-scan acquisition is used, because the physical parameter T₁ often is the same for both scans, Eq. 4 can be used to obtain:

Z_{1,k}⁺×( [symbol] )^{TR}^{₁}^{/TR}^{₂}−(Z_{2,k}⁺)^{TR}^{₁}^{/TR}^{₂}× [symbol] =0 [11].

A factor Ω_{i,k} is defined as a scaling factor for a given scan i and pathway k:

Ω_{i,k}=F_{i,k}⁻+F_{i,-k}⁻. [12]

The quantity X_{i,k}, referred to as a ‘mixing factor,’ captures how the +k and −k transverse magnetization states are intermixed after an RF pulse:

$${X}_{i , k} = 1 - \frac{2 \times ( {F}_{i , k}^{-} - {F}_{i , k}^{+} )}{{Ω}_{i , k}} . \qquad [ 13 ] .$$

Because the factors Ω_{i,k} and X_{i,k} involve only transverse magnetization states F that can be measured using a pulse sequence, they are considered known. Equations 6 and 13 can be combined with the trigonometric identity cos(α)=2×cos²(α/2)−1 and with cᵢ=cos(αᵢ) to obtain:

$${Z}_{i , k}^{-} = \frac{{Ω}_{i , k}}{2 \times sin ( {α}_{i} )} \times ( {X}_{i , k} - {c}_{i} ) ; \vert k \vert > 0 , \qquad [ 14 ] .$$

Replacing Eq. 14 into Eq. 8, one obtains:

$${Z}_{i , k}^{+} = \frac{{Ω}_{i , k}}{2 \times sin ( {α}_{i} )} \times ( {X}_{i , k} {c}_{i} - 1 ) ; \vert k \vert > 0. \qquad [ 15 ] .$$

The next step is to derive an equation involving only the cosine values, cᵢ and known quantities such as TRᵢ, Ω_{i,k}, and X_{i,k} by replacing all of the longitudinal Z states in Eq. 11 with their corresponding expressions from Eqs. 14 and 15. In a case where scans are sequential, Z_{i,k} ⁻ is equal to [symbol] once steady-state is reached:

(Sequential acquisition) [symbol] =Z_{i,k}⁻. [16].

Combining Eq. 11 with Eqs. 14-16, one obtains:

### (Sequential Acquisition)

$${( {X}_{1 , k} {c}_{1} - 1 )}^{\frac{{TR}_{2}}{{TR}_{1}}} \times ( {X}_{2 , k} - {c}_{2} ) - ( {X}_{2 , k} {c}_{2} - 1 ) \times {( {X}_{1 , k} - {c}_{1} )}^{\frac{{TR}_{2}}{{TR}_{1}}} = 0 ; \\ \vert k \vert > 0. \qquad [ 17 ] .$$

On the other hand, for interleaved acquisitions, once steady-state is reached, one would have:

(Interleaved) [symbol] =Z_{h(i),k}⁻ [18]

where the h(i) function returns the scan number that differs from i in a two-scan acquisition, i.e., h(1)=2 and h(2)=1. For interleaved acquisitions, combining Eq. 11 with Eqs. 14, 15, and 18, one obtains:

$$\begin{matrix} ( Interleaved ) & \\ {( {Ω}_{1 , k} \times sin ( {α}_{2} ) )}^{( \frac{{TR}_{2}}{{TR}_{1}} + 1 )} \times {( {X}_{1 , k} {c}_{1} - 1 )}^{\frac{{TR}_{2}}{{TR}_{1}}} \times ( {X}_{1 , k} - {c}_{1} ) - {( {Ω}_{2 , k} \times sin ( {α}_{1} ) )}^{( \frac{{TR}_{2}}{{TR}_{1}} + 1 )} \times ( {X}_{2 , k} {c}_{2} - 1 ) \times {( {X}_{2 , k} - {c}_{2} )}^{\frac{{TR}_{2}}{{TR}_{1}}} = 0 ; \vert k \vert > 0. & [ 19 ] .\end{matrix}$$

If TR₁ and TR₂ in the interleaved acquisition are different, k=0 pathway may not be entirely refocused at the time when an RF pulse is applied, and several of the equations above may be inaccurate. Therefore, TR₁=TR₂ for interleaved acquisitions, and Eq. 19 is simplified as to:

Interleaved Ω_{1,k}²×(1−c₂²)×(X_{1,k}c₁−1)×(X_{1,k}−c₁)−Ω_{2,k}²×(1−c₁²)×(X_{2,k}c₂−1)×X_{2,k}−c₂)=0; |k|>0. [20].

Combining Eq. 10 with Eq. 17 or 20 leads to a single-variable equation that can be solved numerically for α₁ for sequential or interleaved acquisitions. This equation may have more than one root; the proper root can be selected based on the calculated T₁ and M₀ values.

To convert Eqs. 17 and 20 into single-variable equations, a link between α₁ and the various cosine cᵢ values expressed in Eq. 10 can be used. When hard pulses (i.e., non-selective RF pulses) or small flip angle pulses are used, the ratio between nominal user-input values, \hat{α}ᵢ/\hat{α}₁, can be accurate although individual values \hat{α}ᵢ may not be:

(Hardpulses or \hat{α}ᵢ<<180°) cᵢ=g(α₁,\hat{α}₁,\hat{α}ᵢ)=cos(\hat{α}ᵢ×α₁/\hat{α}₁) [21].

Selecting widely different \hat{α}₁ and \hat{α}₂ in a two-scan acquisition can have desirable effects on the robustness of the method, but as such at least one of the flip angles is likely to take on values large enough to violate the condition \hat{α}ᵢ, <<180°. Such a case does not apply to hard RF pulses because Eq. 21 applies even with large flip angles. However, sequences with slab-selective RF pulses can be useful, as slab selection can be used to limit the extent of the imaged field-of-view and thus reduce scan time. In the case of slab-selective pulses of large flip angles, a Bloch equation simulation of the RF pulse may be performed to numerically establish a function g at each location \vec{r} within the imaged field of view such that:

c₂=g(α₁, \hat{α}₁, \hat{α}₂, \vec{r}). [22],

Note, for large pulses, the FDA limits on specific absorption rate (SAR) of the RF pulses may not be a hurdle because TR values are relatively long (tens of ms) and schemes, such as VERSE, can be used to lower the SAR.

T₁ and M₀ are evaluated as follows. Different scans and different pathways provide independent opportunities to evaluate T₁ and M₀, and a way to identify the correct root for α₁ when necessary. T₁ and M₀ values are calculated for each root, each scan and pathway, and the root providing the best consistency across scans and pathways is selected. T₁ and M₀ values calculated with the correct a, root are then combined through a weighted sum performed over scans and pathways, to improve SNR and robustness.

Combining Eq. 4 with Eqs. 14-16, one obtains for sequential acquisitions:

$$\begin{matrix}( Sequential acquisition ) & \\ {T}_{1 , i , k} = {TR}_{i} / ln ( \frac{{Z}_{i , k}^{+}}{{Z}_{i , k}^{⇒}} ) = {TR}_{i} / ln ( \frac{{X}_{i , k} \times {c}_{i} - 1}{{X}_{i , k} - {c}_{i}} ) ; \vert k \vert > 0. & [ 23 ] .\end{matrix}$$

All independently-obtained T₁ values can be combined through a weighted sum:

$${T}_{1} = \frac{{\sum }_{i , k} {w}_{i , k} \times {T}_{1 , i , k}}{{\sum }_{i , k} {w}_{i , k}} , \qquad [ 24 ] ;$$

where the weights, w_{i,k}, can be chosen to capture the fact that acquisitions with longer TR settings or with stronger signal levels are expected to lead to more reliable T₁ and M₀ measurements, and that only scans acquired with full spatial resolution may be used to obtain a full spatial resolution T₁ map. The expression w_{i,k}=| [symbol] −Z_{i,k}⁻| may be employed toward choosing the weights.

Using the k=0 case from Eqs. 4 and 6 along with Eqs. 8, 16 and 24, as well as the fact that F_{i,0}⁻ represents refocused magnetization and can be assumed to be real-valued without loss of generality, one obtains:

$$\begin{matrix}( Sequential acquisition ) & \\ {M}_{0 , i , k} = \vert \frac{( ( {F}_{i , 0}^{+} - {c}_{i} \times {F}_{i , 0}^{-} ) + ( {F}_{i , 0}^{-} - {c}_{i} \times {F}_{i , 0}^{+} ) \times {e}^{- \frac{{TR}_{i}}{{T}_{1}}} )}{\sqrt{1 - {c}_{i}^{2}} \times ( 1 - {e}^{- \frac{{TR}_{i}}{{T}_{1}}} )} \vert , & [ 25 ] ;\end{matrix}$$

where the identity sin(αᵢ)=±\sqrt{1−cos²(αⱼ)} is used to remove dependency on the sine of αᵢ. Given that M₀ is positive, the ambivalence in sign is resolved by applying an absolute value operator.

As expressed in Eqs. 16 and 18, [symbol] differs for sequential and interleaved acquisitions. So does the expression for T₁:

$$\begin{matrix}( Interleaved ) & \\ {T}_{1 , i , k} = \vert \pm {TR}_{i} / ln ( \frac{{Ω}_{i , k} \times \sqrt{1 - {c}_{h ( i )}^{2}} \times ( {X}_{i , k} {c}_{i} - 1 )}{{Ω}_{h ( i ) , k} \times \sqrt{1 - {c}_{i}^{2}} \times ( {X}_{h ( i ) , k} - {c}_{h ( i )} )} ) \vert ; \\ \vert k \vert > 0 , & [ 26 ] .\end{matrix}$$

For the case k=0, combining Eqs. 4, 6, 8, 18 and 24, one obtains an expression for M₀:

$$\begin{matrix}( Interleaved ) & \\ {M}_{0 , i , k} = \vert \frac{\begin{matrix}( {F}_{h ( i ) , 0}^{+} - {c}_{h ( i )} \times {F}_{h ( i ) , 0}^{-} ) / \\ \sqrt{1 - {c}_{h ( i )}^{2}} + ( {F}_{i , 0}^{-} - {c}_{i} \times {F}_{i , 0}^{+} ) \times {e}^{- \frac{{TR}_{i}}{{T}_{1}}} / \sqrt{1 - {c}_{i}^{2}}\end{matrix}}{( 1 - {e}^{- \frac{{TR}_{i}}{{T}_{1}}} )} \vert . & [ 27 ] .\end{matrix}$$

For each possible root of α₁ and its associated cosine cᵢ values, T_{1,j,k} and M_{0,j,k} are estimated for all available scans and pathways. T_{1,j,k} is evaluated first, from Eq. 23 or 26, using c, along with the known values for TRᵢ, the mixing factors X_{i,k}, and the scaling factors Ω_{i,k}. The combined T₁ value from Eq. 24 is then used to evaluate M_{0,i,k} from Eq. 25 or 27. When there is more than one root, the proper one can be identified, using the fact that the flip angle is close to its nominal value near the center of the FOV and varies smoothly from there.

All of the processing above can be simplified by using real numbers instead of complex numbers. The F and Z magnetization states used throughout the derivations above are complex numbers. The computation can be greatly simplified if real numbers can be used instead.

The longitudinal magnetization can be always flipped into the transverse plane as the transverse magnetization along the same axis, called herein as the x or ‘real’ axis, if the RF pulses have zero phase. Ideally, such sequence would lead to a real signal that refocuses along the x axis only. But imperfections and inhomogeneities in the B₀ and B₁ fields, from a variety of sources, make the measured signals and the corresponding F and Z states complex. For example, the B₀ sensitivity of imaging coils have a spatially-varying phase, and the B₀ field may vary in space and in time due to susceptibility effects and eddy currents. But these various effects tend to affect the phase of F states, not their magnitude. If the phase of all of the F states is ignored and the real values of the F states are used instead, a slew of problems and effects that are not of interest can be avoided. Of course, phase information is valuable. It is extracted and used to calculate frequency offsets for susceptibility-weighted imaging.

Idealized real-valued F states free of phase errors caused by B₀ and B₁ imperfections can be derived as follows. The magnitude of these F states is obtained from the measured signal as in Eq. 3. The correct polarity is given to each F state to convert its magnitude value into a correct real value. The polarity can be determined by noticing that in the absence of RF chopping and 0<αᵢ<180°, the magnetization for all positive pathways should be on the +x axis while that of negative pathways have been inverted and lie on the +x axis:

F_{i,k}^{O}=+F_{i,k}^{O}|, k≧0

F_{i,k}^{O}=−|F_{i,k}^{O}|, k<0 [28];

where O is a placeholder for the ‘+’, ‘−’ or ‘ [symbol] ’ superscripts. In cases where RF chopping is used or a, exceeds 180°, Eq. 28 can still be used without introducing any error or limitation because only the cosine value of the flip angles, the cᵢ values, are needed and recovered. But using Eq. 28 for the cases that RF chopping is used may lead to confusion between αᵢ and −aᵢ, but does not affect T₁ and M₀ measurements as cos(αᵢ)=cos(−αᵢ). Similarly, the use of αᵢ values that exceed 180° can create confusion between αᵢ and (360°−αᵢ), but this does not affect the evaluation of T₁ and M₀ as cos (αᵢ)=cos (360°−αᵢ). Overall, using Eq. 28 makes all processing above real instead of complex and greatly improves the robustness of the method as a whole, especially in the presence of B₀ and B₁ imperfections. These real-valued F states from Eq. 28 can be used in the places of the complex-valued F states to evaluate the T₁, T₂, T₂*, M₀, cosine of the flip angles as described above.

Δf can be evaluated as follows. B₀ field maps can reveal susceptibility differences in tissues. B₀ maps, like T₂* maps, can be generated using a multi-echo gradient-echo sequence. Referring to FIG. 4 again, three echoes (echo 1, 2, and 3) are acquired during one TR. The phase change from echo to echo is divided by the echo spacing to give a frequency value, then is converted into a field value using the gyromagnetic ratio.

The scan time of the system and method as disclosed herein can be further reduced. If two scans are used, a full acquisition uses a scan time of Ny×Nz×(TR₁+TR₂). One source of acceleration can come from shortening the second scan. The second scan, with its large nominal flip angle \hat{α}₂, can be restricted to finding the flip angle map. So only a central region in ky-kz, such as of size 64×64, may be sampled for the second scan. As for the first scan, the scan time can be reduced by parallel imaging and partial k-space sampling. For example, the central 20% along ky and kz can be fully sampled ( 1/25 of k-space volume) while outer regions can be accelerated by a 2-fold acceleration with parallel imaging along both the ky and kz directions and use a 75% partial-Fourier scheme.

Therefore, systems and methods have been described for performing quantitative imaging of a subject using a MRI system. The method can include generating a steady-state pulse sequence on the MRI system, performing the pulse sequence to acquire MR signals from a volume of the subject with two or more scans, representing physical parameters of the subject in relation with the MR signals through equations, generating values of the physical parameters of the subject, and generating a report indicating the physical parameters. The pulse sequence can sample two or more magnetization pathways of MR signals and at least one scan parameter of the pulse sequence changes from scan to scan. The equations representing the relation between the physical parameters and the MR signals are linear equations or single-variable equations.

The values of the physical parameters of the subject are generated by solving those equations. In one configuration, such equations can include:

$$ln ( \vert {S}_{i , k} ( {TE}_{i , j , k} ) \vert ) = \{ \begin{matrix}ln ( \vert {S}_{i , k} ( 0 ) \vert ) - {TE}_{i , j , k} \times ( {R}_{2} + {R}_{2}^{′} ) , & k \geq 0 \\ ln ( \vert {S}_{i , k} ( 0 ) \vert ) - {TE}_{i , j , k} \times ( {R}_{2} - {R}_{2}^{′} ) , & k < 0\end{matrix}$$

wherein TE_{i,j,k} represents TE for the ith scan, jth echo, and kth magnetization pathway, S_{i,k}(TE_{i,j,k}) represents the MR signals at TE_{i,j,k}, R₂ and R₂′ represent irreversible and reversible transverse decay, respectively. Real numbers of the MR signals can be used in the above steps.

A report may be generated with those values indicating the physical parameters.

In one configuration, the two or more magnetization pathways may include at least two of a −2nd, a −1st, a 0th, and a +1st magnetization pathway.

In one configuration, after generating a report, the method may include generating parametric maps of the subject with the parametric values derived. Also, the method may include displaying the parametric maps on a displaying unit.

In one configuration, after generating a report, the method may include generating images of the subject in a desired weighting, using the parametric values derived, and further include displaying the images on a displaying unit.

In one configuration, the physical parameters may include at least two of T₂, T₂*, T₁, M₀, flip angle, and frequency offset. The scan parameter of the pulse sequence that changes from scan to scan may include flip angle and TR. The scans can be sequential or interleaved. When interleaved scans are conducted, the TR for each scan may be the same. The pulse sequence may include a slab-selective RF pulse.

In another aspect of the invention, an MRI system is provided that includes a magnet system configured to generate a polarizing magnetic field about at least a portion of a subject arranged in the MRI system and a magnetic gradient system including a plurality of magnetic gradient coils configured to apply at least one magnetic gradient field to the polarizing magnetic field. The MRI system also includes an RF system configured to apply an RF field to the subject and to receive MR signals therefrom. The MRI system includes a computer system programmed to control the gradient system and the RF system to cause the gradient and RF system to generate a steady-state pulse sequence on the MRI system, control the MRI system to perform the pulse sequence to acquire MR signals from a volume of the subject with two or more scans, represent physical parameters of the subject in relation with the MR signals through equations, generate values of the physical parameters of the subject, and generate a report indicating the physical parameters. The pulse sequence can sample two or more magnetization pathways of MR signals and at least one scan parameter of the pulse sequence changes from scan to scan. The equations representing the relation between the physical parameters and the MR signals are linear or single-variable equations. The values of the physical parameters of the subject are generated by solving those equations. Lastly, a report is generated with those values indicating the physical parameters.

In yet another aspect of the invention, methods for performing quantitative imaging of a subject using a MRI system with one scan is provided. The method can include generating a steady-state pulse sequence on the MRI system, performing the pulse sequence to acquire MR signals from a volume of the subject with one scan, representing physical parameters of the subject in relation with the MR signals through equations, generating values of the physical parameters of the subject, and generating a report indicating the physical parameters. The pulse sequence can sample two or more magnetization pathways of MR signals. The equations representing the relation between the physical parameters and the MR signals are linear equations or single-variable equations. The physical parameters include T₂, T₂*, and frequency offset.

The present invention has been described in terms of one or more preferred embodiments, and it should be appreciated that many equivalents, alternatives, variations, and modifications, aside from those expressly stated, are possible and within the scope of the invention.

As used in the claims, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

1. A method for quantitative imaging of a subject using a magnetic resonance imaging (MRI) system, comprising:
a) generating, using the MRI system, a steady-state pulse sequence to sample two or more magnetization pathways of magnetic resonance (MR) signals;
b) performing the pulse sequence to acquire the MR signals from a volume of the subject with two or more scans, wherein
a scan refers to repetitions of the pulse sequence enough times so to scan the volume of the subject once; and
at least one scan parameter of the pulse sequence changes from scan to scan;
c) assembling a series of linear equations or single-variable equations using the MR signals to represent physical parameters of the subject in relation with the MR signals;
d) generating values of the physical parameters of the subject by solving the linear equations or single-variable equations; and
e) generating a report indicating the physical parameters.

2. The method as recited in claim 1, wherein the two or more magnetization pathways include at least two of a −2nd, a −1st, a 0th, and a +1st magnetization pathway.

3. The method as recited in claim 1, wherein step e) includes generating parametric maps of the subject with the values of the physical parameters, and further comprising step f) displaying the parametric maps on a displaying unit.

4. The method as recited in claim 1, wherein step e) includes generating images of the subject in a desired weighting using the values of the physical parameters, and further comprising step f) displaying the images on a displaying unit.

5. The method as recited in claim 1, wherein the physical parameters include at least two of T₂, T₂*, T₁, M₀, flip angle, and frequency offset.

6. The method as recited in claim 5, wherein step d) includes solving:
$$ln ( \vert {S}_{i , k} ( {TE}_{i , j , k} ) \vert ) = \{ \begin{matrix}ln ( \vert {S}_{i , k} ( 0 ) \vert ) - {TE}_{i , j , k} \times ( {R}_{2} + {R}_{2}^{′} ) , & k \geq 0 \\ ln ( \vert {S}_{i , k} ( 0 ) \vert ) - {TE}_{i , j , k} \times ( {R}_{2} - {R}_{2}^{′} ) , & k < 0\end{matrix}$$
wherein TE_{i,j,k} represents echo time (TE) for ith scan, jth echo, and kth magnetization pathway, S_{i,k}(TE_{i,j,k}) represents the MR signals at TE_{i,j,k}, and R₂ and R₂′ represent irreversible and reversible transverse decay of the MR signals respectively.

7. The method as recited in claim 1, wherein the scan parameter of the pulse sequence that changes from scan to scan includes flip angle or repetition time (TR).

8. The method as recited in claim 1, wherein the scans are performed sequentially.

9. The method as recited in claim 1, wherein the scans are interleaved and a TR for each scan is consistent.

10. The method as recited in claim 1, wherein the pulse sequence comprises a slab-selective RF pulse.

11. The method as recited in claim 1, wherein real numbers of the MR signals are used in steps c) and d).

12. A magnetic resonance imaging (MRI) system, comprising:
a magnet system configured to generate a polarizing magnetic field about at least a portion of a subject arranged in the MRI system;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance (MR) signals therefrom;
a computer system programmed to:
control the gradient system and the RF system to generate a steady-state pulse sequence to sample two or more magnetization pathways of the MR signals;
control the gradient system and the RF system to perform the pulse sequence to acquire MR signals from a volume of the subject with two or more scans using the pulse sequence, wherein
a scan refers to repetitions of the pulse sequence enough times so to scan the volume of the subject once, and
at least one scan parameter of the pulse sequence changes from scan to scan;
assemble a series of linear equations or single-variable equations using the MR signals to represent physical parameters of the subject in relation with the MR signals; and
generate a report indicating the physical parameters.

13. The system as recited in claim 12, wherein the two or more magnetization pathways include at least two of a −2nd, a −1st, a 0th, and a +1st magnetization pathway.

14. The system as recited in claim 12, wherein the computer is further configured to generate parametric maps of the subject using the physical parameters, and further comprising a display configured to display the parametric maps received from the computer system.

15. The system as recited in claim 12, wherein the computer system is further configured to generate images of the subject in a desired weighting using the physical parameters, and further comprising a display configured to display the images received from the computer system.

16. The system as recited in claim 12, wherein the physical parameters include at least two of T₂, T₂*, T₁, M₀, flip angle, and frequency offset.

17. The system as recited in claim 16, the computer system is further configured to solve the series of linear equations or single-variable equations to determine the physical parameters using:
$$ln ( \vert {S}_{i , k} ( {TE}_{i , j , k} ) \vert ) = \{ \begin{matrix}ln ( \vert {S}_{i , k} ( 0 ) \vert ) - {TE}_{i , j , k} \times ( {R}_{2} + {R}_{2}^{′} ) , & k \geq 0 \\ ln ( \vert {S}_{i , k} ( 0 ) \vert ) - {TE}_{i , j , k} \times ( {R}_{2} - {R}_{2}^{′} ) , & k < 0\end{matrix}$$
wherein TE_{i,j,k} represents echo time (TE) for ith scan, jth echo, and kth magnetization pathway, S_{i,k}(TE_{i,j,k}) represents the MR signals at TE_{i,j,k}, and R₂ and R₂′ represent irreversible and reversible transverse decay of the MR signals respectively.

18. The system as recited in claim 12, wherein the scan parameter of the pulse sequence that changes from scan to scan includes flip angle and repetition time (TR).

19. The system as recited in claim 12, wherein the scans are interleaved and a TR for each scan is consistent.

20. The system as recited in claim 12, wherein the computer system is further configured to use real numbers of the MR signals to assemble the series of linear equations or single-variable equations using the MR signals.

21. A method for quantitative imaging of a subject using a magnetic resonance imaging (MRI) system, comprising:
a) generating, using the MRI system, a steady-state pulse sequence to sample two or more magnetization pathways of magnetic resonance (MR) signals;
b) performing the pulse sequence to acquire the MR signals from a volume of the subject with a scan, wherein
a scan refers to repetitions of the pulse sequence enough times so to scan the volume of the subject once;
c) assembling a series of linear equations or single-variable equations using the MR signals to represent physical parameters of the subject in relation with the MR signals;
d) generating values of the physical parameters of the subject by solving the linear equations or single-variable equations; and
e) generating a report indicating the physical parameters.

22. The method as recited in claim 1, wherein the two or more magnetization pathways include at least two of a −2nd, a −1st, a 0th, and a −1st magnetization pathway.

23. The method as recited in claim 1, wherein step e) includes generating parametric maps of the subject with the values of the physical parameters, and further comprising step f) displaying the parametric maps on a displaying unit.

24. The method as recited in claim 1, wherein step e) includes generating images of the subject in a desired weighting using the values of the physical parameters, and further comprising step f) displaying the images on a displaying unit.

25. The method as recited in claim 1, wherein the physical parameters include T₂, T₂* , and frequency offset.

26. The method as recited in claim 5, wherein step d) includes solving:
$$ln ( \vert {S}_{i , k} ( {TE}_{i , j , k} ) \vert ) = \{ \begin{matrix}ln ( \vert {S}_{i , k} ( 0 ) \vert ) - {TE}_{j , k} \times ( {R}_{2} + {R}_{2}^{′} ) , & k \geq 0 \\ ln ( \vert {S}_{i , k} ( 0 ) \vert ) - {TE}_{j , k} \times ( {R}_{2} - {R}_{2}^{′} ) , & k < 0\end{matrix}$$
wherein TE_{j,k} represents echo time (TE) for jth echo, and kth magnetization pathway, Sₖ(TE_{j,k}) represents the MR signals at TE_{j,k}, and R₂ and R₂′ represent irreversible and reversible transverse decay of the MR signals respectively.

27. The method as recited in claim 1, wherein the pulse sequence comprises a slab-selective RF pulse.

28. The method as recited in claim 1, wherein real numbers of the MR signals are used in steps c) and d).
